(19) United States
(12) Patent Application Publication
(10) Pub. No.: US 2010/0204243 A1
(43) Pub. Date: Aug. 12, 2010

(54) Process for the Preparation of Poly DL-Lactide-Co-Glycolide Nanoparticles Having Antitubercular Drugs Encapsulated Therein

(75) Inventors: Gopal Krishan Khuller, Chandigarh (IN); Rajesh Pandey, Chandigarh (IN); Sadhna Sharma, Chandigarh (IN); Jitendra Nath Verma, Haryana (IN)

Correspondence Address: THE WEBB LAW FIRM, P.C., 700 KOPPERS BUILDING, 436 SEVENTH AVENUE, PITTSBURGH, PA, 15219, US

(73) Assignee: LIFECARE INNOVATIONS PVT. LTD., Gurgaon , Haryana (IN)

(21) Appl. No.: 11/918,080

(22) PCT Filed: Apr. 11, 2005
(86) PCT No.: PCT/IN2005/000108

§ 371 Date: Feb. 23, 2010

(51) Int. Cl. A61K 31/496; A61P 31/06

(52) U.S. Cl. 514/254.11

(57) ABSTRACT

A process for the preparation of Poly DL-lactide-co-glycolide nanoparticles having antitubercular drugs encapsulated therein comprising: (i) preparation of an aqueous solution of stable water soluble drugs in DW/NS/PBS (ii) preparation of unstable drugs in DW/NS/PBS (iii) preparation of a polymer and hydrophobic drug solution in an organic solvent, (iv) mixing separately the solutions of steps (i) and (ii) with that of step (iii) and sonicating under cold conditions, (v) adding the above emulsion to aqueous PVA and resonicating under cold conditions, (vi) stirring the emulsion 0nd centrifuging the same (vii) washing the said particles, reconstituting the same and lyophilizing.

### FIELD OF INVENTION

This invention relates to a preparation of poly DL-lactide-co-glycolide nanoparticles (PLG-NP) having an active substance or substances (ATD) encapsulated therein and such that the encapsulated substances are stable with respect to each other. In particular, this invention relates to a preparation comprising first encapsulated active substances which are stable and second encapsulated active substances which are unstable with respect to said first substance. Reference to active substances is intended to include therapeutic and/or bioactive substances. Thus, and as by way of example, the nanoparticles may be employed for encapsulation of antitubercular drugs (ATD).

### BACKGROUND OF INVENTION

The need to administer multiple AID daily for 6-9 months is responsible for patient non-compliance as well as drug-related hepatotoxicity, which result in therapeutic failure. Another consequence of incomplete/irregular treatment is the emergence of drug resistance.

Nanotechnology based drug delivery systems employing, biodegradable polymers have been extensively studied over the past decade. Of the several procedures available to prepare nanoparticles such as double-emulsion-solvent-evaporation, solvent diffusion in oil, microemulsion, gas antisolvent precipitation, gelification of anionic polysaccharides etc., none is perfect in terms of particle size, drug encapsulation efficiency and drug release kinetics. Further, multidrug encapsulation in single formulation is not yet reported.

The most commonly employed double emulsion-solvent-evaporation method involves 2 major steps—formation of droplets in the primary emulsion and subsequent removal of solvent from the droplets of the secondary emulsion followed by polymer precipitation. Particle stability as well as drug release kinetics is controlled by using emulsifiers/stabilizers such as polyvinyl alcohol (PVA).

It is generally known that INH, PZA and RIF are active substances or drugs employed for the treatment of tuberculosis. Thus, patent application no. 765/Del/2003 suggests a process for the simultaneous or co-encapsulation of two or more of the aforesaid drugs, but which are stable with respect to each other.

Besides, the aforesaid three drugs or active substances, it is also known that EMB is a drug which is also employed for the treatment of tuberculosis. However, EMB is unstable in the presence of the INH, PZA or RIF, and particularly in the presence of INH. Thus, it has been found that EMB could not be coencapsulated simultaneously with INH, as any such coencapsulation would result in a degradation of EMB. Thus, patent application no. 765/Del/95 had a useful application for coencapsulation of active substances or drugs which were compatible to each other with respect to stability. The encapsulation of EMB was not hitherto known.

### OBJECTS OF THE INVENTION

An object of this invention is to propose a preparation and a process there for containing encapsulated active substance, which are unstable with respect to each other.

Another object of this invention is to propose a preparation and a process there for, having ATD encapsulated therein and obviates the disadvantages associated with the known art, and wherein ATD comprises a combination of either rifampicin (RIF)+isoniazid (INH)+pyrazinamide (PZA)+ethambutol (EMB), or rifampicin (RIF)+isoniazid (INH).

Yet another object of this invention Is to propose a preparation and a process there for, having ATD encapsulated therein which provides a prolonged and sustained drug(s) release.

Still another object of this invention is to propose a preparation and a process there for, having ATD encapsulated therein capable of being modulated to entrap maximum drug.

A further object of this invention is to propose a preparation and a process there for, having ATD encapsulated therein capable of distributing the drug(s) evenly to different organs where tubercle bacteria reside.

A still further object of this invention is to propose a preparation and a process there for, having ATD encapsulated therein which can be lyophilized and reconstituted for use as an oral formulation.

Yet a further object of this invention is to propose a preparation and a process there for, having AID encapsulated therein which does not exhibit hepatotoxicity.

### BRIEF DESCRIPTION OF THE INVENTION

According to this invention there is provided a process for the preparation of Poly DL-lactide-co-glycolide nanoparticles having antitubercular drugs encapsulated therein comprising:
(i) preparation of an aqueous solution of stable water soluble drugs in DW/NS/PBS.
(ii) preparation of unstable drugs in DW/NS/PBS
(iii) preparation of a polymer and hydrophobic drug solution in an organic solvent,
(iv) mixing separately the solutions of steps (i) and (ii) with that of step (iii) and sonicating under cold conditions,
(v) adding the above emulsion to aqueous PVA and resonicating under cold conditions,
(vi) stirring the emulsion and centrifuging the same.
(vii) washing the said particles, reconstituting the same and lyophilizing.

In accordance with this invention, an aqueous solution of hydrophilic drug is prepared in DW/NS/PBS in the ratio of 1:0.1-100 weight by volume. A solution of polymer is prepared in an organic solvent preferably dichloromethane (DCM) in the ratio 1:0.3-1 weight by volume, also containing the hydrophobic drug in the ratio 1:0.5-5 weight by volume. The aqueous solution is poured into the organic solution in the ratio 1:5-20 volume by volume and sonicated for 45-120 sec at 4°-15° C. The primary emulsion is poured into 0.8-2.5% PVA solution keeping DCM:PVA ratio at 1:0.5-1.5, sonicated for 2-5 min at 4°-15° C. and stirred for 18-30 hr. The stirred mixture is centrifuged at 8000-12000 rpm for 15-30 min at 4°-20° C. to obtain the pellet and washed 3-4 times with DW/NS/PBS, resuspended in same and lyophilized. The ratio of drug and polymer is kept at 1:1 w/w. In this manner, three types of formulations are prepared, i.e. PLG-NP encapsulating RIF+INH+PZA, PLG-NP encapsulating RIF+INH, and PLG-NP encapsulating EMB.

It has been found that ethambutol is highly unstable in the presence of isoniazid. Thus, if an encapsulation of four active substances is required or an encapsulation of two or more active substances is required and of which ethambutol is one of the active substance, it would have been convenient to coencapsulate ethambutol in conjunction with the other active substances, and employing a process as described in copending patent application no. 765/Del/2003. However, a disadvantage associated therewith is that due to its unstability properties, ethambutol would degrade.

Thus, in order to obviate such a disadvantage, ethambutol is encapsulated separately. Such a separate encapsulation of ethambutol also improves the bioavailability of ethambutol upon oral administration. Yet another property is that of minimum inhibitory concentration (MIC). It has been found that a MIC is not achieved with a four active substance encapsulation with ethambutol being one of the active substances. However, a separate encapsulation of ethambutol provides the required MIC.

A process for the preparation of PLG-NP having ATD encapsulated is explained by the following example.

### EXAMPLE

10mg INH and 10 mg PZA were dissolved in 1 mL DW. 10 mg RIF and 30 mg PLG were suspended in 10 mL DCM. The aqueous solution was added to the DCM solution, sonicated at 4° C. for 1 min and poured into 1% PVA solution (8 mL) followed by sonication at 4° C. for 3 min. The emulsion was stirred for 18 hr and centrifuged at 10,000 rpm for 20 min. The pellet was washed 3 times with DW and then resuspended in the same for lyophilization.

10 mg EMB was dissolved in 1 mL DW. 10 mg PLG was suspended in 10 ml DCM. The aqueous solution was added to the DCM solution, sonicated at 4° C. for 1 min and poured into 1% PVA solution (8 mL) followed by sonication at 4° C. for 3 min. The emulsion was stirred for 18 hr and centrifuged at 10,000 rpm for 20 min. The pellet was washed 3 times with DW and then resuspended in the same for lyophilization.

Lyophilized particles were suspended in NS and administered orally to mice and the results are given in Table 1.

TABLE 1
Colony forming units (CFUs) of M. tuberculosis in organs of mice
after drug treatment.

| | | | | Log cfu/organ | |
|---|---|---|---|---|---|
| | | | Number | Lungs | |
| Groups | Dose | Schedule | of doses | Spleen | |
| 1. Untreated | — | — | — | 4.97 ± 0.07 | 4.91 ± 0.09 |
| control | | | | | |
| 2(a) Free 3-drugs | Therapeutic | Daily | 28 | 2.88 ± 0.09 | 2.85 ± 0.06 |
| combination | | | | | |
| (b) PLG 3-drugs | Therapeutic | Every 10 days | 3 | 2.75 ± 0.06 | 2.75 ± 0.10 |
| combination | | | | | |
| (c) PLG 3-drugs | ½-Therapeutic | Every 7 days | 4 | 2.72 ± 0.05 | 2.78 ± 0.04 |
| combination | | | | | |
| 3(a) Free 4-drugs | Therapeutic | Daily | 28 | <1.00* | <1.00* |
| combination | | | | | |
| (b) PLG 4-drugs | Therapeutic | Every 10 days | 3 | <1.00* | <1.00* |
| combination | | | | | |
| 4(a) Free 4-drugs | ½-Therapeutic | Daily | 28 | 2.67 ± 0.14 | 2.72 ± 0.07 |
| combination | | | | | |
| (b) PLG 4-drugs | ½-Therapeutic | Every 7 days | 4 | <1.00* | <1.00* |
| combination | | | | | |

Values are mean ± SD, n = 5 per group
The log cfu values were comparable (P > 0.05) between Groups 2 a/b/c and 4(a), which were, however, significantly lower (P < 0.001) than Group 1.
*Indicates no detectable cfu on day 28 following the inoculation of undiluted and 1 in 10 diluted tissue homogenates.

A single oral administration of PLG nanoparticles to mice, the minimum inhibitory concentration (MIC for ethambutol=1.5 μg/ml) was achieved in the plasma only when EMB was encapsulated and administered separately. This is important from the point of view of treatment of TB because if the drug levels are below MIC, the treatment becomes ineffective. In fact, when PLG-NP co-encapsulating EMB along with other 3 drugs were administered to mice, EMB levels in the blood were below MIC throughout the study period.

Furthermore, with reference to free EMB whose bioavailability is considered to be=1, the bioavailability of PLG-NP-encapsulated EMB (alone) was=10.6, whereas, the bioavailability of PLG-NP-encapsulated EMB (along with other 3 drugs) was 5.1.

### Determination of Drug Content in PLG-NP

The drug encapsulation efficiency for PLG-NP were as under:

| | |
|---|---|
| RIF | 56.99 ± 2.72% |
| IHN | 66.31 ± 5.83% |
| PZA | 68.02 ± 5.58% |
| EMB | 43.11 ± 4.21% |

The PLG-NP did not include any hepatotoxicity as assessed by plasma bilirubin, alanine transaminase and alkaline phosphatase.

We claim:

1. A process for the preparation of Poly DL-lactide-co-glycolide nanoparticles having antitubercular drugs encapsulated therein comprising:
(i) preparation of an aqueous solution of stable water soluble drugs in DW/NS/PBS.
(ii) preparation of unstable drugs in DW/NS/PBS
(iii) preparation of a polymer and hydrophobic drug solution in an organic solvent,
(iv) mixing separately the solutions of steps (i) and (ii) with that of step (iii) and sonicating under cold conditions,
(v) adding the above emulsion to aqueous PVA and resonicating under cold conditions,
(vi) stirring the emulsion and centrifuging the same.
(vii) washing the said particles, reconstituting the same and lyophilizing.

2. A process as claimed in claim 1 wherein said organic solvent is dichloromethane.

3. A process as claimed in claims 1 and 2 wherein said polymer is dissolved in said solvent in the ratio of 1:0.3-1 w/v.

4. A process as claimed in claim 1 wherein said drug is dissolved in DW/NS/PBS in the ratio of 1:1-1000 w/v and in DCM in the ratio 1:0.5-5 w/v.

5. A process as claimed in claim 1 wherein said aqueous and organic solutions are mixed in the ratio 1:5-20 v/v.

6. A process as claimed in claim 1 wherein said drug and polymer ratio is 1:1 w/w.

7. A process as claimed in claim 1 wherein first sonication is carried out for 45-120 sec and the second sonication for 2-5 min, each at 4° C.-15° C.

8. A process as claimed in claim 1 wherein said polyvinyl alcohol is of the strength of 0.8-2.5% and DCM:PVA ratio is 1:0.5-1.5.

9. A process as claimed in claim 1 wherein centrifugation is carried out at 8000-12000 rpm for 15-30 min at 4°-20° C.

10. A process as claimed in claim 1 wherein said particles are washed 3-4 times with DW/NS/PBS (pH 7.2-7.4) and lyophilized.

11. A process for the preparation of poly DL-lactide-co-glycolide nanoparticles having antitubercular drugs encapsulated therein substantially as herein described and illustrated in the example.
